(19) United States
(12) Patent Application Publication
(10) Pub. No.: US 2023/0310395 A1
(43) Pub. Date: Oct. 5, 2023

(54) TOPICAL ROFLUMILAST COMPOSITIONS AND USES THEREOF

(71) Applicant: Sol-Gel Technologies Ltd., Ness Ziona (IL)

(72) Inventors: Moshe ARKIN, Kfar Shmaryahu (IL); Ofer TOLEDANO, Kfar Saba (IL); Karine NEIMANN, Ness Ziona (IL); Hila HAKAK DJERBI, Tel Aviv (IL)

(73) Assignee: Sol-Gel Technologies Ltd., Ness Ziona (IL)

(21) Appl. No.: 18/022,522

(22) PCT Filed: Aug. 24, 2021
(86) PCT No.: PCT/IL2021/051038

§ 371 Date: Feb. 22, 2023

Related U.S. Application Data
(60) Provisional application No. 63/069,848, filed on Aug. 25, 2020.
(60) Provisional application No. 63/181,338, filed on Apr. 29, 2021.

(51) Int. Cl. A61K 31/44; A61K 9/00; A61K 9/06; A61K 47/26; A61K 47/10; A61K 47/18; A61K 47/32; A61K 47/12; A61K 47/14; A61K 47/06; A61P 17/00

(52) U.S. Cl. CPC A61K 31/44; A61K 9/0014; A61K 9/06; A61K 47/26; A61K 47/10; A61K 47/183; A61K 47/32; A61K 47/12; A61K 47/14; A61K 47/06; A61P 17/00

(57) ABSTRACT

The present disclosure, in some embodiments, relates to a low permeable topical composition comprising roflumilast, N-oxide of roflumilast or salts thereof, and to a method of treatment of a skin disorder by topical administration of said composition.

### FIELD OF THE INVENTION

The present disclosure, in some embodiments, relates to a low permeable topical composition comprising roflumilast, N-oxide of roflumilast or salts thereof, and to a method of treatment of a skin disorder by topical administration of said composition.

### BACKGROUND OF THE INVENTION

Roflumilast 3-(cyclopropylmethoxy)-n-(3,5-dichloropyridin-4-yl)-4-(difluoromethoxy)-benzamide is a PDE4 inhibitor having the following structure:

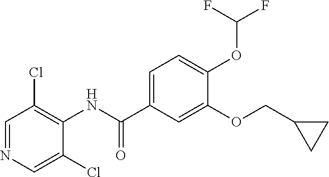 [SMILES: O=C(Nc1c(Cl)cncc1Cl)c1ccc(OC(F)F)c(OCC2CC2)c1]

Compositions containing roflumilast are used in human and veterinary medicine and have been proposed for the treatment and prophylaxis of diseases including but not limited to: inflammatory and allergen-induced airway disorders (e.g. bronchitis, asthma, COPD); dermatoses (e.g. proliferative, inflammatory and allergen induced skin disorders), and generalized inflammations in the gastrointestinal region (Crohn's disease and ulcerative colitis). Roflumilast is a known to be suitable as a bronchial therapeutic agent as well as for the treatment of inflammatory disorders.

The systemic administration of roflumilast is accompanied by side-effects including i.a. emesis and diarrhea, which is a big drawback to the oral administration. The topical administration of roflumilast comprising combination drugs provided in this disclosure avoids the systemic side-effects.

Roflumilast has a very low calculated water solubility (0.0062 mg/mL, according to the roflumilast DrugBank monograph). This low aqueous solubility has been problematic for the development of topical emulsions, suspensions, gels or solutions containing water.

Several approaches have been proposed to improve solubility of roflumilast in parenteral preparations and topical emulsions, suspensions, gels or solutions by including a blend of water-miscible solvents in the pharmaceutical composition (US 2019/091333). This resulted in good permeation and very fast penetration lag time. Unfortunately, such compositions have very high blood levels of roflumilast (US 2019029956). Such blood levels are not recommended as a topical product, especially not with children.

This invention is directed to topical composition comprising high concentration of roflumilast with reduced permeability in order to achieve better efficacy without the risk of unwanted side effects. Preferably, this invention is directed to micronized or coated/encapsulated microcapsules comprising solid roflumilast, salts of roflumilast, the N-oxide of roflumilast or salts thereof.

### SUMMARY OF THE INVENTION

In one embodiment, this invention is directed to a topical composition comprising from about 0.1% w/w to about 2.0% w/w roflumilast, N-oxide of roflumilast, or salts thereof, and a carrier suitable for topical administration, wherein the composition has decreased skin permeability. In other embodiments, the topical composition has a low systemic absorption. In other embodiments, the composition has a skin penetration lag time longer than one hour as measured by in vitro permeation testing (IVPT). In other embodiments, the composition has a skin penetration lag time between 2 hours to 24 hours. In another embodiment, the topical composition has minor penetration to the skin and most of the composition remains on the intact stratum corneum, the outermost layer of epithelium of the skin.

In one embodiment, this invention provides a dosage form comprising a topical composition described herein, wherein said topical composition is formulated as a cream, a lotion, a gel, an ointment, an emulsion, a solution, a suspension, an elixir, a tincture, a paste, a foam, an aerosol, a spray, a patch, a transdermal patch or as a pre-filled applicator syringe.

In one embodiment, this invention provides a method of treatment, prevention or alleviation of an inflammatory skin disorder, said method comprises topical administration to the affected area of a subject with the skin disorder a therapeutically effective amount of a topical composition described herein. In another embodiment, the inflammatory skin disorder is selected from the group consisting of psoriasis (vulgaris), toxic and allergic contact eczema, atopic eczema, seborrheic eczema, lichen simplex, sunburn, pruritus in the genitoanal region, alopecia areata, hypertrophic scars, discoid lupus erythematosus, follicular and extensive pyodermas, acne, rosacea, atopic dermatitis, prurigo nodularis and hidradenitis suppurativa.

In some embodiments, this invention provides a regimen of administration comprising once daily or twice daily administration to an affected area of a subject with an inflammatory skin disorder a therapeutically effective dose of a topical composition as described herein until the skin disorder is cured, prevented or alleviated.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present invention provides a topical composition comprising from about 0.1% w/w to about 2.0% w/w roflumilast, N-oxide of roflumilast or salts thereof, wherein the composition has minor penetration to the skin or having a skin penetration with long lag time; and methods of treatment, prevention and amelioration of a skin disorder.

The present invention provides a topical composition comprising from about 0.1% w/w to about 2.0% w/w roflumilast, N-oxide of roflumilast, or salts thereof, and a carrier suitable for topical administration, wherein the composition has decreased skin permeability. In other embodiments, the topical composition has a low systemic absorption. In other embodiments, the composition has a skin penetration lag time longer than one hour as measured by in vitro permeation testing (IVPT). In other embodiments, the composition has a skin penetration lag time is between 2 hours to 24 hours. In other embodiments, the composition has a skin penetration lag time is between 2 hours to 24 hours as measured by in vitro permeation testing (IVPT). In other embodiments, the composition has minor or negligible permeation capacity through the skin within the first hour of administration.

A drug penetrates the skin into the vasculature then undergoes distribution into the tissues, metabolism and excretion characteristic of the drug further delaying detection in the blood and extending lag time. The lag time measured using in vitro permeation testing (IVPT) is shorter than the lag time measured in PK experiments, because achieving measurable blood levels of active always takes longer than for active to diffuse to the depth in the skin required to reach the vasculature for outflux from the skin. The lag time depends on the compound penetrating the skin and may be an hour or more. Skin penetration enhancers, excipients combined with the pharmaceutical active to formulate a topical product, can influence lag time as well as increase the amount of active crossing the stratum corneum.

The present invention provides a topical composition comprising from about 0.1% w/w to about 2.0% w/w roflumilast, N-oxide of roflumilast or salts thereof, wherein the composition is in the form of dispersion.

In one embodiment, provided herein a topical composition comprising from about 0.1% w/w to about 2.0% w/w roflumilast, N-oxide of roflumilast or salts thereof, wherein the roflumilast is dispersed in a gel.

In one embodiment, provided herein a topical composition comprising from about 0.1% w/w to about 2.0% w/w roflumilast, N-oxide of roflumilast or salts thereof, wherein the roflumilast is dissolved in a gel.

In one embodiment, provided herein a topical composition comprising from about 0.1% w/w to about 2.0% w/w roflumilast, N-oxide of roflumilast or salts thereof, wherein the composition is formulated as a gel. In certain embodiments, the composition is formulated as a gel, wherein the roflumilast is dispersed or fully or partially dissolved therein.

In one embodiment, provided herein a topical composition comprising from about 0.1% w/w to about 2.0% w/w roflumilast, N-oxide of roflumilast or salts thereof, wherein the composition is formulated as a cream and roflumilast is dissolved therein.

In one embodiment, the composition provided herein in a form of a dispersion or a dispersion in a gel, having a skin penetration with longer lag time than the dissolved roflumilast in a cream.

In one embodiment, the composition provided herein in the form of a dispersion.

In one embodiment, the composition provided herein in the form of a dispersion in a gel, or as a fully or partially dissolved roflumilast in a gel, which contains high concentration of roflumilast without an elevation of the systemic penetration.

In one embodiment, the composition is as described in Examples 4 or 5.

In one embodiment, the topical composition provided herein comprising from about 0.1% w/w to about 2.0% w/w roflumilast, N-oxide of roflumilast or salts thereof, in a form of a dispersion having a lower systemic penetration in comparison to a non-dispersed composition.

In some embodiments, this invention provides a topical composition comprising from about 0.1% w/w to about 2.0% w/w roflumilast, N-oxide of roflumilast, or salts thereof, wherein the roflumilast, N-oxide of roflumilast or salts thereof is fully or partially dissolved by a solvent, and said composition possesses less permeation capacity than formulations disclosed in the prior art.

### Topical Composition

In one embodiment, this invention is directed to a topical composition comprising from about 0.1% w/w to about 2.0% w/w roflumilast, N-oxide of roflumilast or salts thereof, wherein the composition has a skin penetration lag time longer than one hour as measured by in vitro permeation testing (IVPT), and a carrier suitable for topical administration.

In another embodiment, the topical composition of this invention has a skin penetration lag time of between 2-24 hrs. In another embodiment, the lag time is between 1-5 hours. In another embodiment, the lag time is between 2-10 hours. In another embodiment, the lag time is between 5-15 hours. In other embodiments, the composition has minor or negligible permeation capacity through the skin within the first hour of administration.

In one embodiment, this invention is directed to a topical composition comprising from about 0.1% w/w to about 2.0% w/w roflumilast, N-oxide of roflumilast or salts thereof, wherein the composition has minor penetration to the skin, and a carrier suitable for topical administration.

In one embodiment, this invention is directed to a topical composition comprising from about 0.1% w/w to about 2.0% w/w roflumilast, N-oxide of roflumilast or salts thereof, wherein the composition has minor penetration to the skin and most of the composition remains on the intact stratum corneum, the outermost layer of epithelium of the skin.

In some embodiment, the topical composition comprises roflumilast, the N-oxide of roflumilast or salts thereof, wherein the roflumilast, N-oxide of roflumilast or salts thereof within the composition is in a solid form.

In some embodiment, the topical composition comprises roflumilast, N-oxide of roflumilast or salts thereof, wherein the roflumilast, N-oxide of roflumilast or salts thereof within the roflumilast is in a suspension.

In some embodiment, the topical composition comprises roflumilast, N-oxide of roflumilast or salts thereof, wherein the roflumilast is dispersed in gel.

In some embodiment, the topical composition comprises roflumilast, N-oxide of roflumilast or salts thereof, wherein the roflumilast is dissolved (partially or fully) in a gel.

In some embodiment, the topical composition comprises roflumilast, N-oxide of roflumilast or salts thereof, wherein the roflumilast, N-oxide of roflumilast or salts thereof is dissolved in cream.

In one embodiment, this invention is directed to a topical composition comprising from about 0.1% w/w to about 2.0% w/w roflumilast, N-oxide of roflumilast or salts thereof, wherein the composition has a skin penetration lag time longer than one hour as measured by in vitro permeation testing (IVPT), and a carrier suitable for topical administration, wherein the roflumilast, salts of roflumilast, the N-oxide of roflumilast or salts thereof is micronized. In other embodiments, the composition has minor or negligible permeation capacity through the skin within the first hour of administration.

In one embodiment, this invention is directed to a topical composition comprising from about 0.1% w/w to about 2.0% w/w roflumilast, N-oxide of roflumilast or salts thereof, wherein the composition has a skin penetration lag time longer than one hour as measured by in vitro permeation testing (IVPT), and a carrier suitable for topical salts of roflumilast, the N-oxide of roflumilast or salts thereof administration, wherein the roflumilast, salts of roflumilast, the N-oxide of roflumilast or salts thereof is encapsulated. In other embodiments, Examples 2 and 3 provide encapsulated roflumilast compositions.

In some embodiments, the roflumilast, salts of roflumilast, the N-oxide of roflumilast or salts thereof is encapsulated in silica shell.

The advantages of the active agents' encapsulation are on the one hand the improved chemical stability of the active agents in the composition and on the other hand its improved therapeutic effect, including reduced side-effects and sustained-release effect.

In some embodiment, the topical composition described herein comprises an encapsulated/coated roflumilast, salts of roflumilast, the N-oxide of roflumilast or salts thereof. In one embodiment, the roflumilast, roflumilast N-oxide, or pharmaceutically acceptable salt is encapsulated/coated.

According to some embodiments of the present invention, the coated/encapsulated form of the active agent(s) (microcapsule) may be in form of a polymeric microsponge/silica microsphere where the active agent is adsorbed, embedded, impregnated or entrapped in the microsponge/silica microsphere as described for example in U.S. Pat. Nos. 4,690,825; 5,145,675, 5,879,716, 5,955,109, and 9,452,137 incorporated herein by reference in their entirety. In some embodiments, the encapsulated roflumilast, N-oxide of roflumilast or salts thereof are prepared as disclosed in U.S. Pat. No. 9,687,465 and US 2010/0016443.

In other embodiments, microcapsules are formed by the encapsulation process disclosed in the following publications (herein incorporated by reference): U.S. Pat. Nos. 7,629,394, 9,205,395, US 2015/0328615, US 2014/0186630. Controlled release microcapsules: IN01958CH2007, IN02080CH2007, U.S. Pat. Nos. 4,235,872, 4,670,250, EP 0248531, U.S. Pat. Nos. 4,970,031, 5,238,714, WO9321764, U.S. Pat. No. 5,575,987, WO9420075, US 2004/137031, US 2006/003014, US 2010/180464; which are incorporated herein by reference in their entirety.

In one embodiment, this invention is directed to a topical composition comprising from about 0.1% w/w to about 2.0% w/w roflumilast, N-oxide of roflumilast or salts thereof, wherein the composition has a skin penetration lag time longer than one hour as measured by in vitro permeation testing (IVPT), and a carrier suitable for topical administration, wherein the composition comprises a solid dispersion. In other embodiments, the composition has minor or negligible permeation capacity through the skin within the first hour of administration.

In one embodiment, this invention is directed to a topical composition comprising from about 0.1% w/w to about 2.0% w/w roflumilast, N-oxide of roflumilast or salts thereof, wherein the composition comprises a dispersion or a suspension. In another embodiment, the composition comprises a solvent which prevent penetration of the composition to the skin. In another embodiment, the composition comprises a non-aqueous solvent.

In another embodiment, the composition comprising this invention is directed to a topical composition comprising from about 0.1% w/w to about 2.0% w/w roflumilast, N-oxide of roflumilast or salts thereof, wherein the composition has a skin penetration lag time longer than one hour as measured by in vitro permeation testing (IVPT), and a carrier suitable for topical administration, wherein the composition comprises a solid dispersion of roflumilast, N-oxide of roflumilast or salts thereof. In other embodiments, the composition has minor or negligible permeation capacity through the skin within the first hour of administration.

In one embodiment, this invention is directed to a topical composition comprising from about 0.1% w/w to about 2.0% w/w roflumilast, N-oxide of roflumilast or salts thereof, wherein the composition has a skin penetration lag time longer than one hour as measured by in vitro permeation testing (IVPT), and a carrier suitable for topical administration. In another embodiment, the topical composition comprises from about 0.3% w/w to about 1.0% w/w roflumilast, salts of roflumilast, the N-oxide of roflumilast or salts thereof. In another embodiment, the topical composition comprises from about 0.5% w/w to about 2.0% w/w roflumilast, salts of roflumilast, the N-oxide of roflumilast or salts thereof. In another embodiment, the topical composition comprises 0.1% w/w, 0.15% w/w, 0.2% w/w, 0.3% w/w, 0.5% w/w, 0.6% w/w, 0.7% w/w, 0.8% w/w, 0.9% w/w, 1.0% w/w, 1.1% w/w, 1.2% w/w, 1.3% w/w, 1.4% w/w 1.5% w/w, 1.6% w/w, 1.7% w/w, 1.8% w/w, 1.9% w/w 2.0 w/w or any ranges thereof of roflumilast, salts of roflumilast, the N-oxide of roflumilast or salts thereof. Each represent a separate embodiment.

The topical composition and methods of use of this invention comprise roflumilast, N-oxide of roflumilast or salts thereof. The topical composition and methods of this invention provide reduced side effects or no side effects of roflumilast, salts of roflumilast, the N-oxide of roflumilast or salts thereof, upon administration. The side effects of roflumilast, salts of roflumilast, the N-oxide of roflumilast or salts thereof include burning, stinging and/or allergic contact dermatitis.

The term “Roflumilast” as used herein includes roflumilast free base, its pharmacologically active metabolites, salts of roflumilast, the N-oxide of roflumilast or salts thereof. Preferably, roflumilast is roflumilast free base. Roflumilast present in the topical composition according to present invention can be in crystalline form or amorphous form.

Suitable salts for roflumilast include inorganic and organic acids. In other embodiments, non-limited examples of acids include: hydrochloric acid, hydrobromic acid, phosphoric acid, nitric acid, sulphuric acid, acetic acid, citric acid, D-gluconic acid, benzoic acid, 2-(4-hydroxybenzoyl)benzoic acid, butyric acid, sulphosalicylic acid, maleic acid, lauric acid, malic acid, fumaric acid, succinic acid, oxalic acid, tartaric acid, embonic acid, stearic acid, toluenesulphonic acid, methanesulphonic acid or 3-hydroxy-2-naphthoic acid, the acids being employed in salt preparation of roflumilast.

### Dosage Form

In some embodiments, this invention comprises a dosage form comprising a topical composition comprising from about 0.1% w/w to about 2.0% w/w roflumilast, salts of roflumilast, the N-oxide of roflumilast or salts thereof, wherein the composition has minor penetration to the skin, and a carrier suitable for topical administration, wherein said composition is formulated as a cream, a lotion, a gel, an ointment, an emulsion, a solution, a suspension, an elixir, a tincture, a paste, a foam, an aerosol, a spray, a patch, a transdermal patch and a pre-filled applicator syringe.

In some embodiments, this invention comprises a dosage form comprising a topical composition comprising from about 0.1% w/w to about 2.0% w/w roflumilast, salts of roflumilast, the N-oxide of roflumilast or salts thereof, wherein the composition has a skin penetration lag time longer than one hour as measured by in vitro permeation testing (IVPT), and a carrier suitable for topical administration, wherein said composition is formulated as a cream, a lotion, a gel, an ointment, an emulsion, a solution, a suspension, an elixir, a tincture, a paste, a foam, an aerosol, a spray, a patch, a transdermal patch and a pre-filled applicator syringe. In other embodiments, the composition has minor or negligible permeation capacity through the skin within the first hour of administration.

In another embodiment, the dosage form is a cream. In another embodiment, the dosage form is a lotion. In another embodiment, the dosage form is a gel. In another embodiment, the dosage form is an ointment. In another embodiment, the dosage form is an emulsion. In another embodiment, the dosage form is a solution. In another embodiment, the dosage form is a suspension. In another embodiment, the dosage form is an elixir. In another embodiment, the dosage form is a tincture. In another embodiment, the dosage form is a paste. In another embodiment, the dosage form is a foam. In another embodiment, the dosage form is an aerosol. In another embodiment, the dosage form is a spray. In another embodiment, the dosage form is a patch. In another embodiment, the dosage form is a transdermal patch. In another embodiment, the dosage form is a pre-filled applicator syringe.

The present invention provides a topical composition comprising from about 0.1% w/w to about 2.0% w/w roflumilast, N-oxide of roflumilast, or salts thereof, and a carrier suitable for topical administration, wherein the composition has decreased skin permeability. In other embodiments, the topical composition has a low systemic absorption. In other embodiments, the composition has a skin penetration lag time longer than one hour as measured by in vitro permeation testing (IVPT). In other embodiments, the composition has a skin penetration lag time is between 2 hours to 24 hours. In other embodiments, the composition is formulated as a cream, wherein the roflumilast, N-oxide of roflumilast or salt thereof is dissolved therein. In other embodiments, the composition has minor or negligible permeation capacity through the skin within the first hour of administration.

The present invention provides a topical composition comprising from about 0.1% w/w to about 2.0% w/w roflumilast, N-oxide of roflumilast, or salts thereof, and a carrier suitable for topical administration, wherein the composition has decreased skin permeability. In other embodiments, the topical composition has a low systemic absorption. In other embodiments, the composition has a skin penetration lag time longer than one hour as measured by in vitro permeation testing (IVPT). In other embodiments, the composition has a skin penetration lag time is between 2 hours to 24 hours. In other embodiments, the roflumilast, N-oxide of roflumilast or salt thereof is fully or partially dissolved in the dosage form. In another embodiment, the dosage form is a gel.

The present invention provides a topical composition comprising from about 0.1% w/w to about 2.0% w/w roflumilast, N-oxide of roflumilast, or salts thereof, and a carrier suitable for topical administration, wherein the composition has decreased skin permeability. In other embodiments, the topical composition has a low systemic absorption. In other embodiments, the composition has a skin penetration lag time longer than one hour as measured by in vitro permeation testing (IVPT). In other embodiments, the composition has a skin penetration lag time is between 2 hours to 24 hours. In other embodiments, the roflumilast, N-oxide of roflumilast or salt thereof is formulated as a dispersion.

The present invention provides a topical composition comprising from about 0.1% w/w to about 2.0% w/w roflumilast, N-oxide of roflumilast, or salts thereof, and a carrier suitable for topical administration, wherein the composition has decreased skin permeability. In other embodiments, the topical composition has a low systemic absorption. In other embodiments, the composition has a skin penetration lag time longer than one hour as measured by in vitro permeation testing (IVPT). In other embodiments, the composition has a skin penetration lag time is between 2 hours to 24 hours. In other embodiments, the composition has minor or negligible permeation capacity through the skin within the first hour of administration. In other embodiments, the roflumilast, N-oxide of roflumilast or salt thereof is dispersed or dissolved in a gel.

The present invention provides a topical composition comprising from about 0.1% w/w to about 2.0% w/w roflumilast, N-oxide of roflumilast, or salts thereof, wherein the roflumilast, N-oxide of roflumilast or salts thereof is fully or partially dissolved by a solvent, and said composition possesses less permeation capacity than formulations disclosed in the prior art.

In some embodiments, the roflumilast, N-oxide of roflumilast, or salts thereof within the compositions of this invention is fully or partially dissolved by a solvent. In other embodiments the solvent comprises DMSO, isosorbid dimethyl ether, PEG 400, propylene glycol, diethyl sebacate, oleyl alcohol, ethanol, transcutol, methoxypolyethylene glycol 350, diisopropyl adipate or mixtures thereof. In other embodiment, roflumilast, N-oxide of roflumilast or salts thereof is fully or partially dissolved by DMSO. In other embodiment, roflumilast, N-oxide of roflumilast or salts thereof is fully or partially dissolved by isosorbid dimethyl ether. In other embodiment, roflumilast, N-oxide of roflumilast or salts thereof is fully or partially dissolved by PEG 400. In other embodiment, roflumilast, N-oxide of roflumilast or salts thereof is fully or partially dissolved by propylene glycol. In other embodiment, roflumilast, N-oxide of roflumilast or salts thereof is fully or partially dissolved by diethyl sebacate. In other embodiment, roflumilast, N-oxide of roflumilast or salts thereof is fully or partially dissolved by oleyl alcohol. In other embodiment, roflumilast, N-oxide of roflumilast or salts thereof is fully or partially dissolved by ethanol. In other embodiment, roflumilast, N-oxide of roflumilast or salts thereof is fully or partially dissolved by transcutol. In other embodiment, roflumilast, N-oxide of roflumilast or salts thereof is fully or partially dissolved by methoxypolyethylene glycol 350. In other embodiment, roflumilast, N-oxide of roflumilast or salts thereof is fully or partially dissolved by diisopropyl adipate.

In some embodiments, the topical composition of this invention possesses less permeation capacity compared to ARQ-151 (topical roflumilast cream formulation of Arcutis) or ARQ-154 (topical roflumilast foam formulation of Arcutis).

In another embodiment, the composition provided herein is a gel, wherein the roflumilast, N-oxide of roflumilast or salt thereof is dispersed within the gel having a longer skin penetration lag time than ARQ-151.

In another embodiment, the composition provided herein is a gel, wherein the roflumilast, N-oxide of roflumilast or salt thereof is dispersed within the gel having longer skin penetration lag time than ARQ-154.

Pharmaceutical carriers or vehicles suitable for preparation of the compositions provided herein include any such carriers known to those skilled in the art to be suitable for the particular mode of administration.

The resulting topical composition may be a lotion, a solution, a suspension, an emulsion or the like and is formulated as creams, gels, ointments, emulsions, solutions, elixirs, lotions, suspensions, tinctures, pastes, foams, aerosols, sprays, patches, foams or any other formulation suitable for topical administration. The preferred compositions are the cream, the gel and the lotion.

Pharmaceutical carriers or vehicles suitable for administration of the active agent provided herein include any such carriers known to those skilled in the art to be suitable for the particular mode of administration. In addition, roflumilast is formulated as the sole pharmaceutically active agent in the composition. The active agent is included in the carrier in an amount sufficient to exert a therapeutically useful effect i.e., amelioration of the symptoms of the skin disorder, with minimal or no toxicity or other side effects. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream and hydrophilic ointment.

Suitable pharmaceutically and dermatologically acceptable vehicles for topical application include lotions, creams, solutions, foam, gels, tapes and the like. Generally, the vehicle is either organic in nature or an aqueous emulsion and capable of having the selected compound or compounds, which may be micronized, dispersed, suspended or dissolved therein. The vehicle may include pharmaceutically-acceptable emollients, moisturizers, including lactic acid, ammonium lactate and urea, coloring agents, fragrances, emulsifiers, thickening agents, vegetable oils, essential oils, zinc oxide and solvents.

### Skin Disorders Treated with the Topical Compositions of this Disclosure

The skin disorders treated with the topical compositions of this disclosure are selected from psoriasis (vulgaris), toxic and allergic contact eczema, atopic eczema, seborrheic eczema, lichen simplex, sunburn, pruritus in the genitoanal region, alopecia areata, hypertrophic scars, discoid lupus erythematosus, follicular and extensive pyodermas, acne, rosacea, atopic dermatitis, prurigo nodularis and hidradenitis suppurativa. The acne is selected from acne vulgaris, papulopustular acne and nodular acne, and the rosacea is selected from erythematotelangiectatic rosacea, papulopustular rosacea, rhinophyma rosacea and ocular rosacea.

### Acne

Acne is a chronic inflammatory disease of the pilosebaceous unit resulting from androgen-induced increased sebum production, altered keratinization, inflammation, and bacterial colonisation of hair follicles on the face, neck, chest, and back by Propionibacterium acnes (P. acnes). Although early colonisation with P. acnes and family history might have important roles in the disease, exactly what triggers acne and how treatment affects the course of the disease remains unclear (Williams H. C. et al., The Lancet, Vol.379. January 2012, pp. 361-372).

There is no ideal treatment for acne. Good quality evidence on comparative effectiveness of common topical and systemic acne therapies is scarce. Topical therapies including benzoyl peroxide, retinoids, and antibiotics when used in combination usually improve control of mild to moderate acne, but suffer from side-effects. Treatment with combined oral contraceptives can help women with acne. Patients with more severe inflammatory acne usually need oral antibiotics combined with topical benzoyl peroxide to decrease antibiotic-resistant organisms.

Oral isotretinoin is the most effective therapy and is used early in severe disease, but its use is limited by teratogenicity and other side-effects.

The treatment of acne with the composition of this invention results in reduced side-effects, due to long penetration lag time.

### Rosacea (Acne Rosacea)

Rosacea is a chronic skin disease that affects more than 16 million Americans. The cause of rosacea is still unknown, and there is no cure. However, research has allowed doctors to find ways to treat the condition by minimizing its symptoms (Cynthia Cobb, Healthline May 21, 2018).

There are four subtypes of rosacea. Each subtype has its own set of symptoms. It is possible to have more than one subtype of rosacea at a time.

Rosacea's typical symptom is small, red, pus-filled bumps on the skin that are present during flare-ups. Typically, rosacea affects only skin on the nose, cheeks, and forehead.

Flare-ups often occur in cycles. This means that one will experience symptoms for weeks or months at a time, the symptoms will go away, and then return.

The four types of rosacea are:
Subtype one, known as erythematotelangiectatic rosacea (ETR), is associated with facial redness, flushing, and visible blood vessels.
Subtype two, papulopustular (or acne) rosacea, is associated with acne-like breakouts, and often affects middle-aged women.
Subtype three, known as rhinophyma, is a rare form associated with thickening of the skin on your nose. It usually affects men and is often accompanied by another subtype of rosacea.
Subtype four is known as ocular rosacea, and its symptoms are centered on the eye area.

### Dermatitis (Eczema)

Dermatitis is a group of diseases resulting in skin inflammation, itchiness, red skin and rash. The dermatitis group of diseases includes atopic dermatitis (AD), allergic contact dermatitis, irritant contact dermatitis and stasis dermatitis. Atopic dermatitis is the most common type of dermatitis.

### Prurigo Nodularis (PN)

PN is characterized by hyperkeratotic excoriated papulonodular (itchy) lesions that show symmetrical distribution on the extensor surfaces of the body and/or extremities, the numbers of which range from several to hundreds (Akarsu, S. et al. An Bras Dermatol. 2018;93(5):671-9.). It is a rare disease and is one of the most difficult conditions in terms of determining its etiology and treatment among chronic skin diseases.

### Hidradenitis Suppurativa (HS)

Hidradenitis suppurativa, also known as acne inversa, is a long-term chronic skin disease whose present treatment options are often unsatisfactory. HS has a profound effect on patient's quality of life (QoL). Alavi A. et al., reviewed QoL aspects of this disease in an article titled “Quality-of-Life Impairment in Patients with Hidradenitis Suppurativa” Am J Clin Dermatol. 2015 February; 16(1):61-5

The clinical picture of HS includes solitary nodules, diffuse, painful abscesses, malodorous drainage, sinus tract formation and scarring.

The exact cause of hidradenitis suppurativa is still unclear, but it is believed that the underlying mechanism involves dysfunction of the apocrine sweat glands or hair follicles.

There is no cure for HS, but treatments with drugs selected from oral antibiotics, corticosteroid injections, antiandrogen therapy with high dosages of cyproterone acetate and ethynyl estradiol) TNF inhibitors like adalimumab and immunosuppressive drugs have been attempted.

### Methods of Treatment

According to an aspect of the invention, there is provided a method of treatment, prevention or alleviation of an inflammatory skin disorder of a subject in need thereof, wherein the method comprises administering a topical composition from about 0.1% w/w to about 2.0% w/w roflumilast, salts of roflumilast, the N-oxide of roflumilast or salts thereof, wherein the composition has minor penetration to the skin, and a carrier suitable for topical administration.

According to an aspect of the invention, there is provided a method of treatment, prevention or alleviation of an inflammatory skin disorder of a subject in need thereof, wherein the method comprises administering a topical composition from about 0.1% w/w to about 2.0% w/w roflumilast, salts of roflumilast, the N-oxide of roflumilast or salts thereof, wherein the composition has a skin penetration lag time longer than one hour as measured by in vitro permeation testing (IVPT), and a carrier suitable for topical administration.

In some embodiments, the method of this invention provides administering a therapeutically effective amount of a composition described herein. In another embodiment, the therapeutically effective amount of the composition refers to an amount which will cure, treat, alleviate or prevent an inflammatory skin disorder.

According to an aspect of the invention, there is provided a method of treatment, prevention or alleviation of an inflammatory skin disorder of a subject in need thereof, wherein the method comprises administering a topical composition from about 0.1% w/w to about 2.0% w/w roflumilast, salts of roflumilast, the N-oxide of roflumilast or salts thereof, wherein the composition has minor penetration the skin, and a carrier suitable for topical administration; wherein the inflammatory skin disorder is selected from the group consisting of psoriasis (vulgaris), toxic and allergic contact eczema, atopic eczema, seborrheic eczema, lichen simplex, sunburn, pruritus in the genitoanal region, alopecia areata, hypertrophic scars, discoid lupus erythematosus, follicular and extensive pyodermas, acne, rosacea, atopic dermatitis, prurigo nodularis and hidradenitis suppurativa.

According to an aspect of the invention, there is provided a method of treatment, prevention or alleviation of an inflammatory skin disorder of a subject in need thereof, wherein the method comprises administering a topical composition from about 0.1% w/w to about 2.0% w/w roflumilast, salts of roflumilast, the N-oxide of roflumilast or salts thereof, wherein the composition has a skin penetration lag time longer than one hour as measured by in vitro permeation testing (IVPT), and a carrier suitable for topical administration; wherein the inflammatory skin disorder is selected from the group consisting of psoriasis (vulgaris), toxic and allergic contact eczema, atopic eczema, seborrheic eczema, lichen simplex, sunburn, pruritus in the genitoanal region, alopecia areata, hypertrophic scars, discoid lupus erythematosus, follicular and extensive pyodermas, acne, rosacea, atopic dermatitis, prurigo nodularis and hidradenitis suppurativa.

In some embodiments, this invention provide a method of treatment, prevention or alleviation of an inflammatory skin disorder of a subject in need thereof, wherein the method comprises administering a topical composition from about 0.1% w/w to about 2.0% w/w roflumilast, salts of roflumilast, the N-oxide of roflumilast or salts thereof, wherein the composition has a skin penetration lag time longer than one hour as measured by in vitro permeation testing (IVPT), and a carrier suitable for topical administration, wherein the treatment, prevention or alleviation reduces the side effects of roflumilast, salts of roflumilast, the N-oxide of roflumilast or salts thereof.

In some embodiment, the methods of this invention comprise treatment, prevention or alleviation of acne. In another embodiment, the acne is selected from acne vulgaris, papulopustular acne and nodular acne.

In some embodiment, the methods of this invention comprise treatment, prevention or alleviation of rosacea. In another embodiment, the rosacea is selected from erythematotelangiectatic rosacea, papulopustular rosacea, rhinophyma rosacea and ocular rosacea.

In some embodiments, this invention provide a method of treatment, prevention or alleviation of an inflammatory skin disorder of a subject in need thereof wherein the treatment, prevention or alleviation comprises once daily or twice daily topical administration to a subject in need thereof of a therapeutically effective amount of a topical composition comprising from about 0.1% w/w to about 2.0% w/w roflumilast, salts of roflumilast, the N-oxide of roflumilast or salts thereof, wherein the composition has a skin penetration lag time longer than one hour as measured by in vitro permeation testing (IVPT) or the composition does not penetrate the skin, and a carrier suitable for topical administration.

In some embodiments, this invention provide a method of treatment, prevention or alleviation of an inflammatory skin disorder of a subject in need thereof wherein the treatment, prevention or alleviation comprises once daily or twice daily topical administration to a subject in need thereof of a therapeutically effective amount of a topical composition comprising from about 0.1% w/w to about 2.0% w/w roflumilast, salts of roflumilast, the N-oxide of roflumilast or salts thereof, wherein the composition has a skin penetration lag time longer than one hour as measured by in vitro permeation testing (IVPT) or the composition does not penetrate the skin, and a carrier suitable for topical administration; wherein the treatment, prevention or alleviation reduces the side effects of roflumilast, salts of roflumilast, the N-oxide of roflumilast or salts thereof.

The frequency of administration of the composition of this invention can be determined empirically. Exemplary frequencies are once daily, twice daily, weekly, bi-weekly and monthly. Typical administration frequencies of the topical combination compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of the skin disorder. For example, dosage administration can begin at from twice a day, to once a day, to two times a week, to once a week, to once every two weeks or less frequent than once every two weeks.

### Regimen of Administration of the Topical Composition

In some embodiments, this invention provide a regimen of administration comprising once daily or twice daily administration to an affected area of a subject with an inflammatory skin disorder a therapeutically effective dose of a topical composition comprising from about 0.1% w/w to about 2.0% w/w roflumilast, salts of roflumilast, the N-oxide of roflumilast or salts thereof, wherein the composition has a skin penetration lag time longer than one hour as measured by in vitro permeation testing (IVPT) or the composition has minor penetration to the skin, and a carrier suitable for topical administration. until the skin disorder is cured, prevented or alleviated.

In some embodiments, this invention provide a regimen of administration comprising once daily or twice daily administration to a subject with a skin disorder a therapeutically effective amount of a dosage form, wherein the dosage form comprises a topical composition comprising from about 0.1% w/w to about 2.0% w/w roflumilast, salts of roflumilast, the N-oxide of roflumilast or salts thereof, wherein the composition has a skin penetration lag time longer than one hour as measured by in vitro permeation testing (IVPT) or the composition has minor penetration to the skin, and a carrier suitable for topical administration, wherein said composition is formulated as a cream, a lotion, a gel, an ointment, an emulsion, a solution, a suspension, an elixir, a tincture, a paste, a foam, an aerosol, a spray, a patch, a transdermal patch and a pre-filled applicator syringe.

Therapeutically effective concentrations of the active agents in the compositions of this invention for treatment, prevention or alleviation of the inflammatory skin disorder are determined by empirical methods known in the art.

The concentration of the active agents in the composition will depend on absorption, inactivation, excretion rates of the active compound, the dosage schedule, and amount administered as well as other factors known to those of skill in the art. The dosage and regimen of administration may be determined by dose finding studies, as known in the art.

Exemplary dosages, strengths and concentrations of roflumilast, salts of roflumilast, the N-oxide of roflumilast or salts thereof in the topical combination compositions of this invention, are in the range of from about or at 0.1% w/w, 0.15% w/w, 0.2% w/w, 0.3% w/w 0.5% w/w, 0.6% w/w, 0.7% w/w, 0.8% w/w, 0.9% w/w, 1.0% w/w, 1.1% w/w, 1.2% w/w, 1.3% w/w, 1.4% w/w 1.5% w/w, 1.6% w/w, 1.7% w/w, 1.8% w/w, 1.9% w/w 2.0 w/w or any ranges thereof of roflumilast, salts of roflumilast, the N-oxide of roflumilast or salts thereof.

Typical administration frequencies of the topical combination compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of the skin disorder. For example, dosage administration can begin at from twice a day, to once a day, to two times a week, to once a week, to once every two weeks or less frequent than once every two weeks.

### DEFINITIONS

Unless defined otherwise, all technical and scientific terms used herein have the same meaning as is commonly understood by one of skill in the art to which the invention pertains. In case of conflict, the specification, including definitions, takes precedence. All patents, patent applications, published applications, articles, publications and other published materials referred to throughout the entire disclosure herein, unless noted otherwise, are incorporated by reference in their entirety.

As used herein “skin permeability” and “permeation capacity” are used herein interchangeably, and refer to the capacity or permeability of roflumilast, N-oxide of roflumilast or salts thereof to penetrate and diffuse through the skin through skin's outermost layer, the stratum corneum and reach the vasculature (blood system). In some embodiments, the compositions of this invention have decreased/low skin permeability. In some embodiments the skin permeability of the roflumilast, N-oxide of roflumilast or salts thereof within the compositions of this invention is of less than 0.3 ng roflumilast/mL of plasma (for 0.15% by weight roflumilast); or less than 0.5 ng roflumilast/mL of plasma (for 0.5% by weight roflumilast). In some embodiments, the skin permeability (or permeation capacity) is lower than a Cmax of 0.187, AUC: 2.82 (0-24 h) (standardized to 1 mg/kg) or lower than a Cmax of 0.126, AUC of 2.43(0-24 h) (standardized to 1 mg/kg).

In other embodiments, the composition disclosed herein has minor or negligible permeation capacity through the skin within the first hour of administration.

As used herein “systemic absorption” refers to the rate and extent at which drugs reach the systemic circulation from the site of administration. In some embodiments, the systemic absorption of roflumilast, N-oxide of roflumilast or salts thereof within the compositions of this invention is less than 1 hour.

As used herein, the indefinite articles “a” and “an” mean “at least one” or “one or more” unless the context clearly dictates otherwise.

As used herein, the term “treating” or “treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

The term “ingredient” refers to a pharmaceutically acceptable ingredient which is included or is amenable to be included in FDA's Inactive Ingredient database (IIG). Inactive ingredients sometimes exhibit some therapeutic effects, although they are not drugs.

As used herein, a “pharmaceutical composition” refers to a composition comprising one or more active ingredients with other components such as pharmaceutically acceptable ingredients or excipients. The purpose of a pharmaceutical composition is to facilitate administration of an active ingredient to a subject.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

As used herein, numerical ranges preceded by the term “about” should not be considered to be limited to the recited range. Rather, numerical ranges preceded by the term “about” should be understood to include a range accepted by those skilled in the art for any given element in formulations according to the present invention.

As used herein, when a numerical value is preceded by the term “about”, the term “about” is intended to indicate ±10%.

The terms “comprise”, “comprising”, “includes”, “including”, “having” and their conjugates mean “including but not limited to”.

The term “consisting of” means “including and limited to”.

The term “consisting essentially of” means that the composition, method formulation may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

### Example 1

### Composition Comprising Roflumilast 1%

Water suspension gel of micronized roflumilast, 1%.

820 g water, 20 g Tween 80 and 160 g roflumilast are milled with a WAB Dynomill for 25 min, while cooling.

1 g of ethylenediaminetetraacetic acid disodium salt (EDTA), 5 g of Imidurea and 5 g of methylparaben (preservatives) are added to 62.5 g of milled roflumilast suspension.

50 g of glycerin are dissolved in 900 kg of water. 10 of Carbopol 980 NF are added and the solution is mixed for 1 hour.

Milled roflumilast is added followed by addition of sodium hydroxide (20%) to form a gel, until pH is in the range of 6-7.

### Example 2

### Composition Comprising Encapsulated Roflumilast

Encapsulation of roflumilast 15% suspension

Three solutions are prepared:
i. An acid solution of 7% w/w citric acid, 10% w/w lactic acid and 32% w/w hydrochloric acid.
ii. 3% Polyquaternium-7 aqueous solution (3 wt. %); and
iii. 27% Sodium silicate solution.

Roflumilast dispersion is prepared by mixing 12 g of roflumilast with 0.4 g CTAC (cetyltrimethylammonium chloride) and 45 g water under high shear.

Preparation of encapsulated roflumilast:
i. Sodium silicate (solution iii-mentioned) is added to the roflumilast dispersion under high shear, followed by adding the acid solution (solution i-mentioned) to adjust the pH to be lower than 6.8, and followed by (adding the Polyquaternium-7 aqueous solution (solution ii-mentioned) to the mixture.
ii. The cycle is repeated 3-10 times at a temperature of between 28° C. to 40° C.
iii. After the final cycle, the pH of the mixture is adjusted to about 5.0 using the acid solution, and water was added to complete the total weight of the mixture to 80 g.

### Example 3

### Composition Comprising Encapsulated Roflumilast

Water suspension gel of encapsulated roflumilast, 0.3%

1 g of ethylenediaminetetraacetic acid disodium salt (EDTA), 5 g of Imidurea and 5 g of methylparaben (preservatives) are added to 18.75 g of encapsulated roflumilast suspension prepared in Example 2.

50 g of glycerin are dissolved in 900 kg of water. 10 of Carbopol 980 NF are added and the solution is mixed for 1 hour.

Encapsulated roflumilast is added followed by addition of sodium hydroxide (20%) to form a gel, until pH is in the range of 6-7.

### Example 4

### Preparation of Roflumilast 0.3% Formulation Dissolved in Cream

TABLE 1

| Raw material | % w/w in the formulation - dissolved in cream |
|---|---|
| Roflumilast | 0.3 |
| Diethyl sebacate | 23 |
| Dimethyl sulfoxide | 3 |
| MYRJ S100 | 1.8 |
| Imwitor 900k | 2.2 |
| Isopropyl myristate | 5 |
| Petrolatum | 10 |
| Cetostearyl alcohol | 3 |
| Cetyl alcohol | 3 |
| Methylparaben | 0.2 |
| Propylparaben | 0.05 |
| Citric acid | For pH adjustment |
| Tri-sodium citrate | For pH adjustment |
| dihydrate | |
| Purified water | q.s to 100 |

### Preparation Procedure for Roflumilast 0.3% Dissolved in Cream

The Roflumilast cream was prepared as follows:
Water phase: Water and buffer were weighed into a glass beaker. MYRJ S100, propylparaben and methylparaben were added. The beaker was placed inside a hot water bath adjusted to 65° C. and mixed with a magnetic stirrer until a clear solution was obtained.
Oil phase: In a separate glass beaker petrolatum, isopropyl myristate, cetostearyl alcohol, cetyl alcohol, and imwitor 900K were weighed-. The beaker was placed inside a hot water bath adjusted to 65° C. and mixed with a magnetic stirrer until a clear solution was obtained.
Active phase: In a separate glass beaker, diethyl sebacate, DMSO and roflumilast were weighed-. The beaker was placed inside a hot water batch adjusted to 65° C. and mixed with a magnetic stirrer until all the powder dissolved and clear solution was formed.

The oil phase was added to the water phase while homogenizing at 5000 rpm for 5 minutes.

Then the active phase was added to the emulsion phase while homogenizing at 5500 rpm for 5 minutes.

The emulsion was cooled down to 25° C., while mixing gently with a mechanical stirrer, until a homogenous cream was obtained.

The cream was discharge and packed into suitable containers.

### Example 5

### Preparation of Roflumilast 0.3% Dispersed in Gel

TABLE 2

| Raw material | % w/w in the formulation - dispersed in gel |
|---|---|
| Roflumilast | 0.30 |
| Tween 80 | 0.06 |
| Propylene glycol | 61.32 |
| Benzoic Acid | 0.10 |
| EDTA | 0.10 |
| Carbopol ®980 | 1.20 |
| NaOH 20% solution | For pH adjustment |
| Purified water | q.s to 100 |

Preparation procedure for Roflumilast suspension was as follows:
In a glass beaker, water, propylene glycol and tween 80 were mixed with a magnetic stirrer until a clear solution was obtained.
Then, roflumilast powder was added and the mixing was continued until the powder was well dispersed.
The mixture was transferred to Dyno-mill and was milled for about 13 minutes at 5500 rpm, until a homogenous suspension was formed.
The suspension was collected and stored at 2-8° C.

Preparation procedure for Roflumilast 0.3% % dispersed in gel

The roflumilast gel was prepared as follows:
In a glass beaker, propylene glycol and benzoic acid were weighed.
The beaker was placed inside a hot water bath adjusted to 65° C. and mixed with a magnetic stirrer until clear solution free from particles was obtained. Then the solution was cooled down to room temperature (25° C.).
In a separate glass beaker, water and EDTA were weighed and mixed with a magnetic stirrer for 15 min until clear solution free form particles was obtained.
Then Carbopol powder was added and dispersed using homogenization at 5000 rpm for 3 min, until all lumps disappeared.
The pH was adjusted with NaOH 20% to pH of about 4.5; until the Carbopol was well neutralized and viscous clear gel was formed.
Then the propylene glycol solution was added while homogenizing at 5000 rpm for 5 minutes until homogenous gel was formed.
The roflumilast suspension was added to gel while homogenizing at 5000 rpm for 5 min until homogenous white gel is formed.
The final pH was measured. Water was added for batch completion and mixed with mechanical stirrer until homogenous viscous gel was obtained.
The gel was discharged and packed into suitable containers.

### Example 6

### Efficacy Study of Roflumilast (as provided herein) for the Treatment of Psoriasis

The objective of this study was to determine the efficacy of Roflumilast as provided herein (at Examples 4 and 5) compared to the vehicle and Dexamethasone as shown at the Table 3. The evaluation was based on histology analysis following application of the respective test formulations.

TABLE 3

| Group | Active ingredient | Dosage form |
|---|---|---|
| 1 | Vehicle | Cream |
| 2 | Dexamethasone 2 mg | Solution |
| 3 | Roflumilast 0.3% | Cream |
| 4 | Roflumilast 0.3% | Gel |

The efficacy study has been conducted on female mice. The total duration of the experiment was 8 weeks, upon receiving of the human skin. Normal skin from healthy volunteers were obtained for grafting. Healthy human abdominal skin pieces with a width of 0.4 mm and surface area of 1.5×1.5 cm were provided. The skin sample was preserved in isotonic saline solution and transplanted within 6-12 hours after skin donation. In addition, blood samples were collected from psoriatic patients having classic plaque psoriasis and not undergoing any treatment. 20 mL blood samples were taken from both males and females psoriatic patients (from psoriatic patients having classic plaque psoriasis and not undergoing any treatment), and peripheral blood mononuclear cells (PBMC) were separated immediately after blood withdrawal.

Mice: Beige-severe combined immunodeficient mice C.B-17/IcrHsdscid-bg (beige-SCID) mice (weight ˜25 g) were included in this study. Mice were monitored twice a week for vital signs such as weight, food intake, coat, eyes, anal genital areas, discharge/soiling, behavior and criteria related to skin transplantation. Normal healthy human donor skin were transplanted onto the beige-SCID mice as previously described (See Keren A. et al., Novel nanosome delivery system combined with siRNA targeting the antimicrobial gene DFB4: a new approach for psoriasis management? Exp Dermatol. 2014; 23:464-465; Bracke S. et al., Targeted silencing of DEFB4 in a bioengineered skin-humanized mouse model for psoriasis: development of siRNA SECosome-based novel therapies. Exp Dermatol. 2014 ;23:199-201; Zaretsky M. et al., Directed evolution of a soluble human IL-17A receptor for the inhibition of psoriasis plaque formation in a mouse model. Chem Biol. 2013 21; 20:202-2; Gilhar A. et al., The beneficial effect of blocking Kv1.3 in the psoriasiform SCID mouse model. J Invest Dermatol. 2011;131:118 124; Keren A. et al., Innate lymphoid cells 3 induce psoriasis in xenotransplanted healthy human skin. J Allergy Clin Immunol. 2018;142:305-308.e6.).

PBMC's from the psoriatic patient's blood were isolated and cultured in the presence of a high dose of IL-2; Prospec ,100 U/mL of media-RPMI 1640,10% human AB serum (Sigma, St. Louis, MO), 1% glutamine, 1% antibiotics (media components; Biological Industries, Kibbutz Beit Haemeck, Israel) , as previously described (Gilhar A. et al., The beneficial effect of blocking Kv1.3 in the psoriasiform SCID mouse model. J Invest Dermatol. 2011;131:118-124; Nousbeck J. et al., IGFBP7 as a potential therapeutic target in Psoriasis. J Invest Dermatol. 2011;131:1767-1770; Schafer P H. et al., Apremilast, a cAMP phosphodiesterase-4 inhibitor, demonstrates anti-inflammatory activity in vitro and in a model of psoriasis. British Journal of Pharmacology 2010; 159:842-855).

Four weeks following the engraftment, each mouse was injected (intradermally) with 1×10⁷ IL-2 enriched allogeneic PBMC's from psoriatic patients (1×10⁷ cells injected/mouse). Cells from different psoriasis patients were equally distributed between treatment groups. Each patient was represented in each treatment group.

Fourteen days following cells injections, the mice were divided into treatment groups (Table 4, see below) and treated according to the timeline indicated below (see Scheme 1). Each transplant was equally topically treated (50 μg) by using a sterile spatula.

Dexamethasone (D2915, Sigma): was freshly prepared for each administration by dilution in saline to final concentration (50 mg/ml). Dexamethasone serves as a positive control and was administered topically twice daily for 14 days following cells injections (at Day 42 following human skin transplantation) until the end of the study (Day 56).

The vehicle was administered topically (50 μg) once daily for 14 days as well.

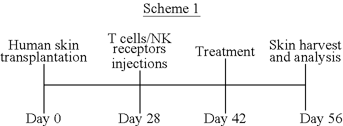

On Day 56, the entire skin graft was excised and placed in 10% formaldehyde in saline overnight. Then, the specimens were placed in 70% ethanol and embedded and stained according to the standard Hematoxylin/Eosin protocol.

Skin graft histological assessment was performed (according to the psoriatic criteria including epidermal thickness) using light microscopy, and two observers blindly performed the evaluations. Epidermal thickness was determined using an ocular micrometer at a minimum of 50 points along the epidermis selected to represent points of maximal and minimal thickness. Thickness of the suprapapillary plate was measured similarly at 50 points for each sample. Three slices were examined for each sample.

### Study Results

TABLE 4
Histological evaluation of psoriasis-induced xenotransplants study:

| | | | Fre- | Psoriatic | Partial | Complete |
|---|---|---|---|---|---|---|
| Group | Compound | Route | quency | Features | Recovery | Recovery |
| 1 | Vehicle | Topical | Once | 9/10 | 1/10 | 0/10 |
| | cream | | daily | (90%) | (10%) | (0%) |
| 2 | Dexa- | Topical | Twice | 0/10 | 1/10 | 9/10 |
| | methasone | | a day | (0%) | (10%) | (90%) |
| | 2 mg | | | | | |
| 3 | Roflumilast | Topical | Once | 6/10 | 1/10 | 3/10 |
| | Dissolved | | daily | (60%) | (10%) | (30%) |
| | in a cream | | | | | |
| | 0.3% | | | | | |
| 4 | Roflumilast | Topical | Once | 4/10 | 0/10 | 6/10 |
| | Dispersed | | daily | (40%) | (0%) | (60%) |
| | in a- Gel | | | | | |
| | 0.3% | | | | | |

Further, Roflumilast 0.3% dispersed in a gel is more potent than Roflumilast 0.3% dissolved in a cream.

1. A topical composition comprising from about 0.1% w/w to about 2.0% w/w roflumilast, N-oxide of roflumilast, or salts thereof, and a carrier suitable for topical administration, wherein the composition has decreased skin permeability.

2. The topical composition according to claim 1, wherein the composition has a low systemic absorption.

3. The topical composition according to claim 1, wherein the composition has a skin penetration lag time longer than one hour as measured by in vitro permeation testing (IVPT).

4. The topical composition according to claim 3, wherein the skin penetration lag time is between 2 hours to 24 hours.

5. The topical composition according to claim 1, wherein the composition has minor penetration to the skin and most of the composition remains on the intact stratum corneum, the outermost layer of epithelium of the skin.

6. The topical composition according to claim 1, wherein the roflumilast, N-oxide of roflumilast or salts thereof within the composition is in a solid form.

7. The topical composition according to claim 1, wherein the roflumilast, N-oxide of roflumilast or salts thereof is micronized.

8. The topical composition according to claim 1, wherein the roflumilast, N-oxide of roflumilast or salts thereof is encapsulated.

9. The topical composition according to claim 1, wherein the composition comprises a solid dispersion.

10. The topical composition according to claim 1, wherein the composition comprises from about 0.3% w/w to about 1.0% w/w roflumilast, N-oxide of roflumilast or salts thereof.

11. The topical composition according to claim 1, wherein the composition comprises from about 0.5% w/w to about 2.0% w/w roflumilast, N-oxide of roflumilast or salts thereof.

12. A dosage form comprising the topical composition of claim 1, wherein said composition is formulated as a cream, a lotion, a gel, an ointment, an emulsion, a solution, a suspension, an elixir, a tincture, a paste, a foam, an aerosol, a spray, a patch, a transdermal patch and a pre-filled applicator syringe.

13. The dosage form of claim 12, wherein the dosage form is a gel, and the roflumilast is dispersed or dissolved within the gel.

14. A method of treatment, prevention or alleviation of an inflammatory skin disorder, said method comprises topical administration to the affected area of a subject with the skin disorder a therapeutically effective amount of a composition of claim 1.

15. The method of claim 14, wherein the inflammatory skin disorder is selected from the group consisting of psoriasis (vulgaris), toxic and allergic contact eczema, atopic eczema, seborrheic eczema, lichen simplex, sunburn, pruritus in the genitoanal region, alopecia areata, hypertrophic scars, discoid lupus erythematosus, follicular and extensive pyodermas, acne, rosacea, atopic dermatitis, prurigo nodularis and hidradenitis suppurativa.

16. The method of claim 15, wherein the inflammatory skin disorder is acne, selected from acne vulgaris, papulopustular acne and nodular acne.

17. The method of claim 15, wherein the skin disorder is rosacea, selected from erythematotelangiectatic rosacea, papulopustular rosacea, rhinophyma rosacea and ocular rosacea.

18. The method of claim 14, wherein the treatment, prevention or alleviation comprises once daily or twice daily topical administration to a subject in need thereof of a therapeutically effective amount of a composition comprising from about 0.1% w/w to about 2.0% w/w roflumilast, N-oxide of roflumilast or salts thereof, and a carrier suitable for topical administration; wherein the composition has minor or negligible permeation capacity through the skin within the first hour of administration.

19. The method of claim 14, wherein the treatment, prevention or alleviation reduces the side effects of roflumilast, N-oxide of roflumilast or salts thereof.

20. A regimen of administration comprising once daily or twice daily administration to an affected area of a subject with an inflammatory skin disorder a therapeutically effective dose of the topical composition of claim 1 until the skin disorder is cured, prevented or alleviated.

21. A regimen of administration comprising once daily or twice daily administration to a subject with a skin disorder a therapeutically effective amount of a dosage form of claim 12.

22. A topical composition comprising from about 0.1% w/w to about 2.0% w/w roflumilast, N-oxide of roflumilast, or salts thereof, wherein the roflumilast, N-oxide of roflumilast or salts thereof is fully or partially dissolved by a solvent, and said composition possesses less permeation capacity than formulations disclosed in the prior art.

23. The topical composition of claim 22, wherein the solvent comprises DMSO, isosorbid dimethyl ether, PEG 400, propylene glycol, diethyl sebacate, oleyl alcohol, ethanol, transcutol, methoxypolyethylene glycol 350, diisopropyl adipate or mixtures thereof.
